(19) United States
(12) Patent Application Publication
(10) Pub. No.: US 2018/0055291 A1
(43) Pub. Date: Mar. 1, 2018

(54) TOOTHBRUSH HOLDER ASSEMBLY

(71) Applicant: Shinwa Kogyo Co., Ltd., Ishikawa (JP)

(72) Inventors: Yasuaki WAZAWA, Ishikawa (JP); Hideyuki WAZAWA, Ishikawa (JP)

(21) Appl. No.: 15/670,336

(22) Filed: Aug. 7, 2017

(30) Foreign Application Priority Data
Aug. 25, 2016 (JP) 2016-164649

(51) Int. Cl. A47K 1/09; A47B 81/02; A61L 2/26; A46B 17/06

(52) U.S. Cl. CPC A47K 1/09; A47B 81/02; B65D 81/24; A46B 17/06; A61L 2/26

(57) ABSTRACT

The invention has for its main object to provide a toothbrush holder assembly that is capable of holding and keeping a toothbrush clean with the head of the tooth-brush upsidedown so that water droplets from the tooth-brush can make direct way down to a washbowl of a washstand, the interior of a kitchen sink, and the floor of a bathroom, and that is capable of preventing the toothbrush held from coming down abruptly on the floor of the bathroom. A toothbrush holder assembly 1 comprises a pedestal 2, a shaft 3 that extends upwardly from the pedestal 2, a holder 4 that is attached in a position of the shaft 3 near the pedestal 2 in such a way as to be rotatable about the axis of the shaft 3, and a support 5 that is attached in a position of the shaft 3 near its upper end in such a way as to be rotatable about the axis of the shaft 3. A toothbrush 6 held by the toothbrush holder assembly 1 comprises a handle 63 that is gripped by the hands of a user, a bristled head 61, and a neck 62 that joins the handle 63 to the head 61. The toothbrush holder assembly 1 is capable of holding the toothbrush 6 with its head 61 placed upside down.

### TECHNICAL FIELD

The present invention relates generally to a toothbrush holder assembly, and more particularly to a toothbrush holder assembly capable of holding tooth-brushes with their heads placed upside down.

### BACKGROUND ART OF THE INVENTION

A toothbrush tends to remain wetted with water because it is usually washed after tooth-brushing. As the toothbrush remaining wetted with water is left in a cup or on a toothbrush rack of a washstand, it may sometimes cause the cup or toothbrush rack to get dirty and insanitary over time.

In addition, moisture and toothpowder remaining between bristles packed at the head of the toothbrush are passed on to the neck and handle, often resulting in re-contamination of them and making the toothbrush itself insanitary.

To avoid such insanitation, it is envisaged that after the toothbrush is fully washed with running water, it is shaken up and down for full straining. In mornings where there are busy activities for going out, however, there would often be no time for sufficient toothbrush washing and straining. Up and down shaking of toothbrushes causes water droplets to hit against mirrors or the like, resulting in the need for wiping out them.

Such problems as mentioned above may be solved by the toothbrush hanger disclosed in Japanese Patent Application Publication No. 2002-238786. This toothbrush hanger includes a hanger body having a magnetizing portion attached to a portion for upside-down suspension of a toothbrush so that a magnet provided via a holder to the end of the toothbrush is sucked onto the magnetizing portion for upside-down suspension of the tooth-brush.

### BRIEF SUMMARY OF THE INVENTION

A problem with such a toothbrush hanger, however, is that water enters between the end of a toothbrush shaft and the holder during tooth brushing or toothbrush washing, often resulting in molding or the like and, hence, insanitation.

A problem with upside-down suspension of the tooth-brush is that a fluid containing molds between the end of the toothbrush shaft and the holder may likely flow toward the head by way of the toothbrush. This may lead to another problem that the head of the toothbrush inserted into the mouth gets insanitary.

Yet another problem with upside-down suspension of the toothbrush is that contact of the magnetizing portion with the toothbrush causes the magnet to run out, resulting in dropping of the toothbrush.

In view of the problems as mentioned above, it is one object of the invention to provide a toothbrush holder assembly that is capable of holding and keeping a toothbrush clean with the head of the tooth-brush placed upside down so that water droplets can make direct way down to a washbowl of a washstand, the interior of a kitchen sink, and the floor of a bathroom, and that is capable of preventing the toothbrush held from abruptly coming down on the floor of the bathroom.

According to one aspect of the invention, the aforesaid object is achievable by the provision of a toothbrush holder assembly capable of holding a toothbrush with its head placed upside down, comprising:

a pedestal,

a shaft that extends upwardly from the pedestal,

a holder mounted in a position of the shaft near the pedestal in such a way as to be rotatable about an axis of the shaft, and including a distal end extending to a position away from the axis of the shaft, on which distal end a portion of the toothbrush can be hooked, said portion increasing in cross-sectional area from a neck to a handle of the toothbrush, and

a support mounted in a position of the shaft near its distal end in such a way as to be rotatable about the axis of the shaft, said support being capable of holding said toothbrush while resting the handle of the toothbrush on said support above the holder.

According to the toothbrush holder assembly of the first aspect of the invention, the toothbrush can be held with its head placed upside down. Accordingly, even when there is no full washing and straining after the use of the toothbrush, moisture and tooth powder remaining between the packed bristles at the head drop directly from the distal end of the head. In other words, moisture and tooth powder remaining between the packed bristles do not run down along the neck and handle so that the toothbrush can be held and kept clean.

According to the toothbrush holder assembly of the first aspect, it is not required to take the trouble to shake the toothbrush up and down because what is needed for full straining of the toothbrush is only to hold and place the toothbrush in the toothbrush holder assembly.

Further according to the toothbrush holder assembly of the first aspect, the portion of the toothbrush increasing in cross-sectional area from the neck to the handle can be hooked on the distal end extending to a position away from the axis of the shaft. As the toothbrush holder assembly is placed such that the head of the toothbrush projects upwardly from the washbowl of a washstand, the interior of a kitchen sink, and the floor of a bathroom, it allows water droplets from the toothbrush to make direct way down to the washbowl of a washstand, the interior of a kitchen sink, and the floor of a bathroom. Even when there is water leakage from the washbowl of a washstand, the interior of a kitchen sink and the floor of a bathroom, they are easily washable with no problem arising.

In addition, according to the toothbrush holder assembly of the first aspect, the portion of the toothbrush increasing in cross-sectional area from the neck to the handle can be hooked on the holder and the handle of the toothbrush is rested against the support above the holder, as described. For this reason, the toothbrush is stably held in the toothbrush holder assembly so that the held toothbrush can be prevented from coming down abruptly.

In the toothbrush holder assembly according to the second aspect of the invention, near the distal end of the holder in the aforesaid holder assembly there is a hole formed for holding an I-type interdental brush with its head placed upside down.

The toothbrush holder assembly according to the second aspect of the invention does not only act as in the toothbrush holder assembly of the first aspect, but also includes an interdental brush holding hole near the distal end of the holder so that water droplets from the I-type interdental brush makes direct way down to the washbowl of a washstand, the interior of a kitchen sink and the floor of a bathroom. Even with leakage of water, the washbowl of a washstand, the interior of a kitchen sink and the floor of a bathroom can be easily washed with no problem arising.

In the toothbrush holder assembly according to the third aspect of the invention, any one of the aforesaid toothbrush holder assemblies further includes a cup rack that is mounted in such a way as to be rotatable about the axis of the shaft so that a cup can be hooked on the cup rack in a position away from the axis of the shaft with its opening placed upside down.

The toothbrush holder assembly according to the third aspect of the invention does not only act as in any one of the aforesaid toothbrush holder assemblies, but also allows a cup to be hooked in a position away from the axis of the shaft with its opening placed upside down. For this reason, hooking of the cup on the cup rack of the toothbrush holder assembly is all that is needed for full straining.

The toothbrush holder assembly according to the third aspect of the invention acts as in any one of the aforesaid toothbrush holder assemblies. In addition, the toothbrush holder assembly is placed such that the opening of the cup projects above the washbowl of a washstand, the interior of a kitchen sink and the floor of a bathroom. Accordingly, water droplets from the cup make direct way down to the washbowl of a washstand, the interior of a kitchen sink and the floor of a bathroom.

According to the toothbrush holder assembly of the fourth aspect of the invention, any one of the aforesaid toothbrush holder assemblies is adjustable in terms of the length to the distal end of the holder extending from the axis of the shaft.

The toothbrush holder assembly according to the fourth aspect of the invention does not only act as in any one of the aforesaid toothbrush holder assemblies, but is also compatible with changes in the length between where the toothbrush holder assembly is set up and the washbowl of a washstand, the interior of a kitchen sink or the floor of a bathroom.

### Advantages of the Invention

Any one of the aforesaid toothbrush holder assemblies is capable of holding and keeping a toothbrush clean with its head placed upside down so that water droplets from the toothbrush can make direct way down to the washbowl of a washstand, the interior of a kitchen sink and the floor of a bathroom and the toothbrush held can be prevented from coming down abruptly.

### BRIEF DESCRIPTION OF DRAWINGS

FIG. 1 is a front view of the toothbrush holder assembly according to one embodiment of the invention.

FIG. 2 is a side view of the toothbrush holder assembly of FIG. 1.

FIG. 3 is a plan view of the toothbrush holder assembly of FIG. 1.

FIG. 4 is a plan view of the toothbrush holder assembly according to another embodiment of the invention.

### DETAILED DESCRIPTION OF THE INVENTION

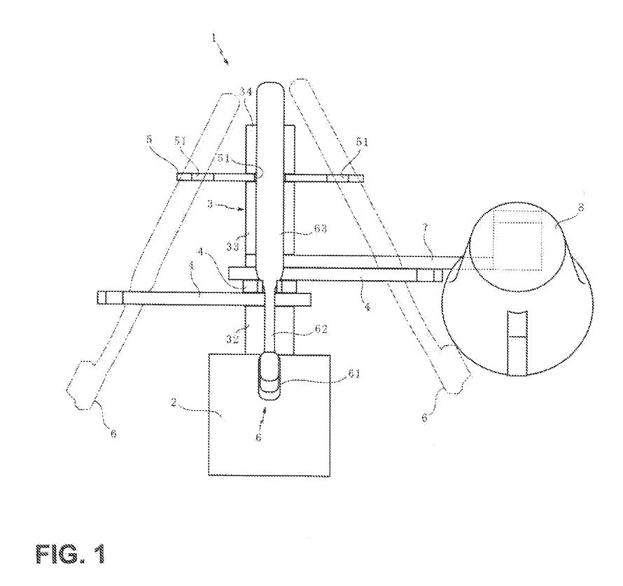

A toothbrush holder assembly 1 according to one embodiment of the invention will now be explained with reference to the accompanying drawings. For an easy understanding of the shape and configuration of the toothbrush holder assembly 1, some toothbrushes 6 are indicated by a two-dot chain line and the shape and configuration of a part of the toothbrush holder assembly 1 are indicated by a solid line in FIGS. 1 to 4.

The toothbrush holder assembly 1 comprises a pedestal 2, a shaft 3 extending upwardly from the pedestal 2, a holder 4 that is attached in a position of the shaft 3 near the pedestal 2 in such a way as to be rotatable about the axis of the shaft 3, and a support 5 that is attached in a position of the shaft 3 near its upper end in such a way as to be rotatable about the axis of the shaft 3.

A toothbrush 6 held by the toothbrush holder assembly 1 comprises a handle 63 gripped by the hand of a user, a bristled head 61, and a neck 62 that links the handle 63 to the head 61.

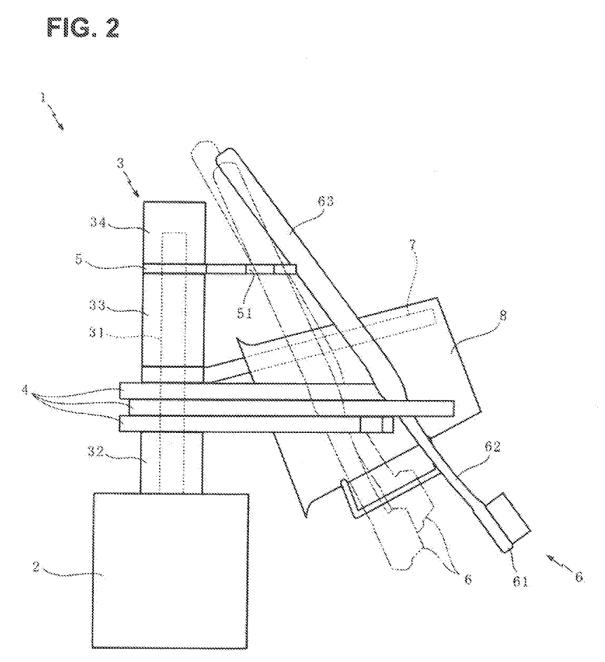

As depicted in FIGS. 1 and 2, the toothbrush holder assembly 1 is capable of holding the toothbrush 6 with its head 61 placed upside down.

As depicted in FIGS. 1, 2 and 3, the pedestal 2 is of round column shape. The pedestal 2 has a weight such that when the bottom of the pedestal 2 is placed on a flat plane, the toothbrush holder assembly 1 does not easily fall down.

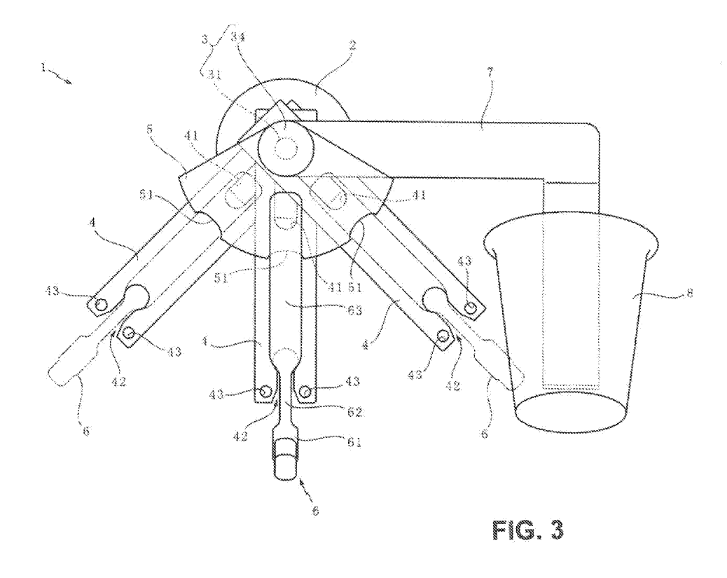

As depicted in FIG. 3, the shaft 3 extends upwardly from the upper surface of the pedestal 2 such that the axis of the round column defining the pedestal 2 and the axis of the shaft 3 are lined up in the same straight line.

As depicted in FIGS. 1 and 2, the shaft 3 comprises a center shaft portion 31, a lower spacer 32, an upper spacer 33 and an axial urge adjustment cap 34. The center shaft portion 31 passes through the lower spacer 32 and upper spacer 33. The axial urge adjustment cap 34 is attached to the upper end of the center shaft portion 31.

As depicted in FIGS. 1 to 3, the holder 4 comprises a plurality of plate members.

As depicted in FIG. 3, near the proximal end of each of the plate members defining the holder 4 there is a slot 41 formed having a longitudinal axis running from the proximal end toward the distal end. Each of the plate members defining the holder 4 is sandwiched, together with a cup rack 7 to be described later, between the lower 32 and the upper spacer 33 while the center shaft portion 31 of the shaft 3 runs through the slot 41.

As the urging action of the lower 32 and the upper spacer 33 in the axial direction is relaxed by the axial urge adjustment cap 34, it causes the plate members of the holder 4 to be rotatable about an axis of the shaft 3. As the axial urging action of the lower 32 and the upper spacer 33 is increased by the axial urge adjustment cap 34, it enables the plate members of the holder 4 to be fixed in place while let out in different directions as shown typically in FIG. 3 and as seen from a top view. Depending on the number of toothbrushes held in place, on the other hand, the holder 4 may be fixed in place while some or all of the plate members are fully put one upon another, as seen from a top view.

The axial urging action of the lower 32 and the upper spacer 33 is relaxed by the axial urge adjustment cap 34, there can be a change in the position of the center shaft portion 31 of the shaft 3 running through the slot 41, resulting in a change in the distance from the axis of the shaft 3 to the distal ends of the plate members defining the holder 4. As the axial urging action of the lower 32 and the upper spacer 33 is increased by the axial urge adjustment cap 34, it enables the holder 4 to be fixed in any desired distance from the axis of the shaft 3 to the distal ends of the plate members of the holder 4.

At the distal ends of the plate members of the holder 4 extending to a position away from the axis of the shaft 2, there is a depression 42 formed from the distal end to the proximal end. As the plate members of the holder 4 are seen from a top view, the depression 42 once becomes narrow from the distal end to the proximal end, and the depth of that narrow depression portion is arched.

As the neck 62 of the toothbrush 6 is inserted into the arched portion, as seen from a top view of the depression 42, it causes a portion of the toothbrush 6 increasing in cross-sectional area from the neck 62 to the handle 63 to be engaged with the arched portion by gravity. That portion of the toothbrush 6 increasing in cross-sectional area may not possibly be disengaged out of the depression 42 because of the presence of the narrow portion of the depression 42. If the toothbrush 6 is lifted up against gravity, the neck 62 of the toothbrush 6 having a small cross-sectional area can then be passed through the narrow portion of the depression 42 so that the toothbrush 6 can be disengaged from the depression 42.

Near the distal ends of the plate members of the holder 4 there is an interdental brush hole 43 capable of holding an I-type interdental brush with its head upside down.

As shown in FIG. 3, the support 5 is a substantially fan-shaped plate member, as seen from a top view. Near a portion of the substantially fan-shaped plate member corresponding to its pivot, as seen from a top view, there is a hole formed. As shown in FIGS. 1 and 2, the support 5 is sandwiched between the upper spacer 33 and the axial urge adjustment cap 34 while the center axis 31 of the shaft 3 runs through this hole. In short, the support 5 is positioned above the holder 4.

A distance from the axis of the shaft 3 to the fan-shaped arc portion, as seen from a top view, of the support 5 is shorter than the distance of the axis of the shaft 3 to the distal depression 42 of each of the plate members of the holder 4 so that the toothbrush 6 can be held by the toothbrush holder assembly 1 in a tilting state, as shown in FIGS. 1 and 2.

The support 5 is provided with a plurality of dents 51 at the arc portion of the substantially fan-shaped configuration as seen from a top view.

The handle 63 of the toothbrush 6 that is rested against the support 5 is in abutment on the dents 51. The dents 51 allow the handle 63 of the toothbrush 6 to be rested against the support 5 in a constant position.

The toothbrush holder assembly 1 further includes a cup rack 7. As shown in FIGS. 1 to 4, the cup rack 7 is attached in such a way as to be rotatable about the axis of the shaft 3. As shown in FIG. 3, the cup rack 7 is a substantially L-shaped plate member, as seen in a top view. The cup rack 7 is provided with a hole on its proximal end side. As shown in FIGS. 1 and 2, the cup rack 7 is sandwiched between the lower 32 and the upper spacer 33 while the center axis 31 of the shaft 3 passes through the proximal end hole in the cup rack 7.

As shown in FIGS. 1, 2 and 3, the cup rack 7 tilts upwardly from the bent portion to the distal end of capital “L” so that a cup 8 can be hooked on the tilting portion of the cup rack 7. The tilting portion of the cup rack 7 allows the cup 8 to be hooked in a position away from the axis of the shaft 3 with the opening of the cup 8 upside down.

In the aforesaid embodiment, the pedestal 2 is described as being of round column shape, but there is no particular limitation on it. The pedestal may be configured as a square column or other cubic shape.

In the aforesaid embodiment, the pedestal 2 may be described as having a weight such that when the bottom of the pedestal 2 is placed on a flat plane, the toothbrush holder assembly 1 does not easily fall down, but there is no particular limitation on it. The pedestal may be provided with a sucker or magnet on its bottom or side periphery.

With a sucker or magnet provided on the bottom of the pedestal, the bottom of the pedestal may be fixed onto a plane on which the toothbrush holder assembly rests to prevent the toothbrush holder assembly from falling down easily. With a sucker or magnet mounted on the side periphery of the pedestal, on the other hand, the pedestal may be attached to a wall surface to prevent the toothbrush holder assembly from falling down easily.

In the aforesaid embodiment, the toothbrush holder assembly is described as comprising the center shaft portion 31, lower spacer 32, upper spacer 33 and axial urge adjustment cap 34, but there is no particular limitation on it. The toothbrush holder assembly may comprise a center shaft portion, three or more spacers and an axial urge adjustment cap. The positions where the holder and support are to be sandwiched are so variable that there can be a change in the height at which toothbrushes are held.

In the aforesaid embodiment, the axial urge adjustment cap 34 is described as being capable of adjusting the urging of the lower spacer 32 and upper spacer 33 in the axial direction, but there is no particular limitation on it. For instance, such axial urging may be adjusted by use of a screw or other means.

In the aforesaid embodiment, the holder 4 is described as comprising three plate members as shown in FIGS. 1, 2 and 3, but there is no particular limitation on it. The holder may comprise two or four or more plate members.

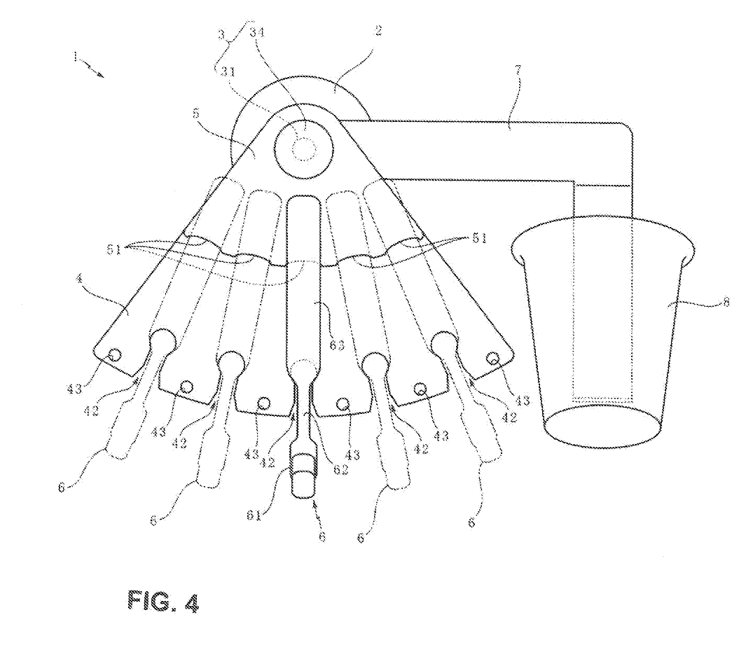

In the aforesaid embodiment, the holder 4 is described as comprising a plurality of plate members, but there is no particular limitation on it. As shown in FIG. 4, the holder 4 may comprise a single plate member of a substantially fan-shaped configuration as seen from a top view. At a portion of this substantially fan-shaped configuration corresponding to its arc portion, as seen from a top view, there are plural depressions 42—explained with reference to the aforesaid embodiment—provided at a given interval. Note here that the reference numerals of FIG. 4 that are the same as in the aforesaid embodiment hold true for those in the aforesaid explanation, and there may be various modifications made to it without being limited to it.

In the aforesaid embodiment, the depression 42 of the holder 4 is described as being configured as shown in FIG. 3 from a top view, but there is no particular limitation on it. The depression of the holder may otherwise be configured provided that a toothbrush can be hooked on it.

In the aforesaid embodiment, the support 5 is described as being formed of a substantially fan-shaped plate member as shown typically in FIG. 3 from a top view, but there is no particular limitation on it. The support may otherwise be configured with the proviso that the handle of a toothbrush can be rested and supported.

In the aforesaid embodiment, the dent of the support 5 is described as being configured as shown in FIGS. 3 and 4, but there is no particular limitation on it. The dent of the support may otherwise be configured with the proviso that it can be rested on the support 5 in a constant position.

### EXPLANATION OF THE REFERENCE NUMERALS

1: Toothbrush holder assembly
2: Pedestal
3: Shaft
4: Holder
5: Support
6: Toothbrush
7: Cup rack
8: Cup
31: Center shaft portion
32: Lower spacer
33: Upper spacer
34: Axial urge adjustment cap
41: Slot
42: Depression
43: Interdental brush hole
51: Dent
61: Head
62: Neck
63: Handle

What is claimed is:

1. A toothbrush holder assembly capable of holding a toothbrush with its head turning downwardly, characterized by comprising,
a pedestal,
a shaft that extends upwardly from the pedestal,
a holder mounted in a position of the shaft near the pedestal in such a way as to be rotatable about an axis of the shaft, and including a distal end extending to a position away from the axis of the shaft, on which a portion of the toothbrush can be hooked, said portion increasing in cross-sectional area from a neck to a handle of the toothbrush, and
a support mounted in a position of the shaft near its distal end in such a way as to be rotatable about the axis of the shaft, said support being capable of holding said toothbrush while resting the handle of the toothbrush on said support above the holder.

2. A toothbrush holder assembly according to claim 1, characterized in that near the distal end of the holder there is a hole provided, said hole being capable of holding an I-type interdental brush with a head of the interdental brush placed upside down.

3. A toothbrush holder assembly according to claim 1, characterized by further including a cup rack which is rotatable about the axis of the shaft and on which a cup can be hooked in a position away from the axis of the shaft with an opening of the cup placed upside down.

4. A toothbrush holder assembly according to claim 2, characterized by further including a cup rack which is rotatable about the axis of the shaft and on which a cup can be hooked in a position away from the axis of the shaft with an opening of the cup placed upside down.

5. A toothbrush holder assembly according to claim 1, characterized in that a length of the holder extending from the axis of the shaft to the distal end of the holder is adjustable.

6. A toothbrush holder assembly according to claim 2, characterized in that a length of the holder extending from the axis of the shaft to the distal end of the holder is adjustable.

7. A toothbrush holder assembly according to claim 3, characterized in that a length of the holder extending from the axis of the shaft to the distal end of the holder is adjustable.

8. A toothbrush holder assembly according to claim 4, characterized in that a length of the holder extending from the axis of the shaft to the distal end of the holder is adjustable.
